(19) United States
(12) Patent Application Publication
(10) Pub. No.: US 2014/0257070 A1
(43) Pub. Date: Sep. 11, 2014

(54) PROCESSING OF LAP SIGNALS

(71) Applicants: Andreas Blomqvist, Taby (SE); Kjell Noren, Solna (SE)

(72) Inventors: Andreas Blomqvist, Taby (SE); Kjell Noren, Solna (SE)

(73) Assignee: ST. JUDE MEDICAL AB, Jarfalla (SE)

(21) Appl. No.: 13/517,009

(22) PCT Filed: Jun. 20, 2011
(86) PCT No.: PCT/EP2011/060200

§ 371 Date: Dec. 27, 2012

(51) Int. Cl. A61B 5/02; A61B 5/04; A61B 5/042; A61B 5/0215; A61B 5/0472

(52) U.S. Cl. CPC A61B 5/02028; A61B 5/0215; A61B 5/0472; A61B 5/0422; A61B 5/04012
USPC 600/374; 600/486

(57) ABSTRACT

Cardiac valve events are monitored by recording a left atrial pressure (LAP) representing signal using an implantable pressure sensor (50). The LAP signal is processed in order to generate a derivative LAP signal representative of the first time derivative of the LAP signal. The opening of the aortic valve of the heart is then identified to coincide in time with a minimum in the derivative LAP signal following ventricular depolarization in a cardiac cycle.

### TECHNICAL FIELD

The embodiments generally relate to processing of left atrial pressure signals, and in particular to identifying specific heart valve events from such left atrial pressure signals.

### BACKGROUND

The heart is the organ that is responsible for circulating blood throughout the body. It consists of four chambers: the left and right atria and the left and right ventricles. In a cardiac cycle, low oxygenated blood that has circulated through the body enters the right atrium and flows through the tricuspid valve into the right ventricle. Simultaneously, oxygenated blood from the lungs enters the left atrium and flows through the mitral or bicuspid valve into the left ventricle. This basically corresponds to the diastolic phase of the cardiac cycle. During the following systolic phase, the ventricles contract forcing low oxygenated blood from the right ventricle through the pulmonary valve towards the lungs and pushing the oxygenated blood from the left ventricle through the aortic valve and into the blood system.

The timing of the opening and closure of the heart valves is generally of diagnostic interest and can be used to detect or monitor various disorders and cardiac conditions. Traditionally, phonograms have been recorded for patients in order to record valve-related heart sounds. In particular, such a phonogram can register the first heart tone, S₁, which corresponds to the closure of the atrioventricular valves, i.e. the mitral and tricuspid valves. A second heart tone, S₂, corresponds to the closure of the semilunar valves, i.e. the aortic and pulmonary valves. Such phonograms are typically recorded using an electronic stethoscope. The closure and opening of heart valves can also be visually monitored in echocardiograms, i.e. cardiac ultrasounds.

Both these prior art techniques of monitoring the opening and closure of the heart valves typically require the patient to visit a physician and conduct the monitoring at a healthcare facility. However, in order to detect various trends and potential deterioration of the cardiac status, it would be advantageous to continuously or periodically monitor the heart valve events over time. This is not possible with the prior art phonogram or echocardiogram recording equipment.

US 2006/0224204 discloses an implantable medical device (IMD) that uses real-time left atrial pressure (LAP) signals obtained from a patient's heart as feedback control mechanism to adjust one or more device parameters of the IMD. The IMD identifies specific characteristics and attributes of the LAP signal that correlate to hemodynamic performance and adjusts the device parameters to optimize the hemodynamic performance. The document suggests that the atrioventricular pacing delay of a dual-chamber pacing IMD can be controlled based on time intervals of the so-called V-wave, A-wave and/or C-wave characteristics in the LAP signal.

There is, though, still a need for an efficient technique that can be used to monitor various cardiac events, such as heart valve events, in real time.

### SUMMARY

It is a general objective to determine cardiac events in an efficient way.

It is a particular objective to identify the timing of heart valve events for a subject in an efficient way.

These and other objectives are met by embodiments disclosed herein.

Briefly, an aspect of the embodiments defines a system for determining cardiac events. The system comprises a connector that is connectable to an implantable pressure sensor. The pressure sensor is configured to generate a left atrial pressure (LAP) signal representative of the left atrial pressure of a subject, preferably a human subject, during at least one cardiac cycle. The system also comprises a derivative processor that processes the LAP signal by calculating a derivative LAP signal representative of the first time derivative of the LAP signal. An aortic valve opening identifier is implemented in the system to process the derivative LAP signal and identify a time point of opening of an aortic valve of the subject's heart based on the derivative LAP signal. The aortic valve opening identifier in particular identifies the aortic valve opening to coincide in time with the time point of a minimum in the derivative LAP signal following ventricular depolarization during a cardiac cycle.

Another aspect of the embodiments relates to a method for identifying cardiac valve events. The method comprises generating a LAP signal representative of a left atrial pressure of a subject during at least one cardiac cycle. A derivative LAP signal is calculated as the first time derivative of the LAP signal. The time point of opening of an aortic valve in the subject's heart is then identified to coincide with the time point of a minimum in the derivative LAP signal following ventricular depolarization during a cardiac cycle.

### BRIEF DESCRIPTION OF THE DRAWINGS

The invention, together with further objects and advantages thereof, may best be understood by making reference to the following description taken together with the accompanying drawings, in which:

FIG. 1 is a schematic block diagram of a system for determining cardiac events according to an embodiment;

FIG. 2 is a schematic block diagram of a system for determining cardiac events according to another embodiment;

FIG. 3 is an average LAP-signal recorded for a subject;

FIG. 4 is an intracardiac electrogram (IEGM) signal recorded for the subject;

FIG. 5 is an average LAP-signal recorded for the subject and an average dLAP/dt signal calculated for the subject;

FIG. 6 schematically illustrates the correlation between pre-ejection period (PEP) calculated according to the embodiments based on a LAP signal (PEP_{LAP}) and PEP obtained from echocardiography data (PEP_{ECHO}) recorded for a subject;

FIG. 7 illustrates an embodiment of processing a LAP signal and an IEGM signal;

FIG. 8 illustrates a system for determining cardiac events at least partly implanted in a human subject;

FIG. 9 is a schematic block diagram of an implantable medical device according to an embodiment; and

FIG. 10 is a flow diagram illustrating a method for identifying cardiac valve events according to an embodiment.

### DETAILED DESCRIPTION

Throughout the drawings, the same reference numbers are used for similar or corresponding elements.

The present embodiments generally relate to the processing of left atrial pressure (LAP) signals and LAP-representing signals in order to identify the timing of certain cardiac events, in particular heart valve related events, in a cardiac cycle.

Traditionally, LAP signals have mainly been employed to calculate and monitor the average LAP as a diagnostic parameter in order to detect congestive heart failure and titrate drugs, in particular diuretics, for heart failure (HF) patients.

The embodiments are instead based on the fact that the LAP signal waveform carries relevant diagnostic information that can be used, with correct processing of the LAP signal waveform, to identify characteristic heart valve related events.

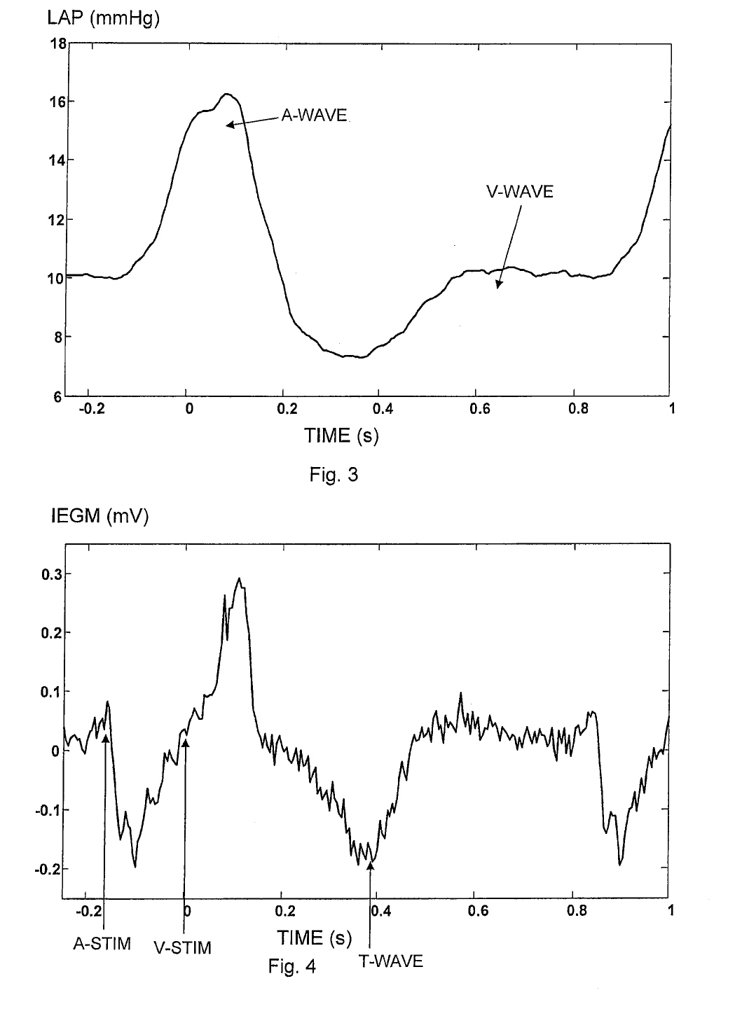

In order to provide more insight in the valuable information that can be extracted from the LAP signal waveform it may be valuable to first describe how the LAP signal waveform changes during a cardiac cycle. FIG. 3 shows an example of an LAP signal waveform recorded for a HF patient. FIG. 4 illustrates the corresponding intracardiac electrogram (IEGM) recorded from the atrial septum for the HF patient.

The diagrams presented in FIGS. 3-6 have been obtained from eight ambulant patients (age 66±12 years and ejection fraction 30±10%) implanted with a CRT-D and an LAP monitoring system (PROMOTE™ RF and HeartPOD™ ISL, St. Jude Medical) and undergoing CRT interval optimization.

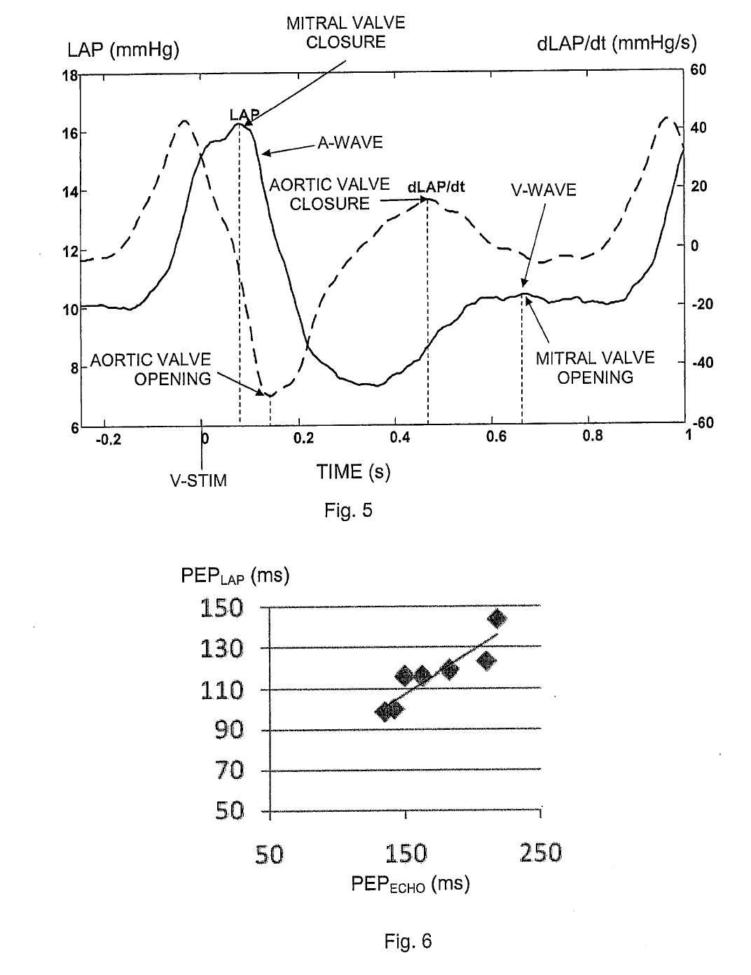

In a cardiac cycle, atrial depolarization causes the atria to contract. The atrial depolarization can be due to intrinsic depolarization or by application of an atrial pacing pulse (A-stim). The contraction of the atria caused by the atrial depolarization results in a rise in the atrial pressure, which is expressed as a positive slope of a so-called A-wave in the LAP signal waveform. The atrioventricular electrical connection causes an intrinsic ventricular depolarization, or the ventricles can be stimulated with a ventricular pacing pulse (V-stim). The ventricular depolarization causes the ventricles to contract, the atrioventricular valves, i.e. the mitral and tricuspid valves, close and the atrioventricular (AV) plane of the heart is pulled downwards towards the heart apex. In addition, the atria are filled with blood from the lungs and from the body. As the AV plane is pulled down by the contracting ventricles there is a dramatic decrease in atrial pressure, seen as the negative slope of the A-wave in FIG. 3. At this point the atrial volume is increasing. During the reduced ejection phase, i.e. still mechanical systole, the atria continue to get filled with blood through venous return. The LAP signal waveform has now passed both its maximum negative derivative and its local minimum value, see FIG. 5, following the A-wave. The LAP signal waveform starts to rise again as the atria bulge with increased venous return. This corresponds to the positive slope of a so-called V-wave. During ventricular repolarization (T-wave) the semilunar valves, i.e. the aortic and pulmonary valves, close marking the end of mechanical systole. The LAP signal waveform still increases slightly due to venous return. The atrioventricular valves then open and blood leaves the atria. The LAP signal waveform has now peaked in the V-wave and following the atrioventricular valve opening the negative slope of the V-wave follows as blood leaves the atria and into the ventricles, thereby resulting in a decrease of atrial pressure. The patient from which the LAP signal illustrated in FIGS. 3-5 is recorded had impaired filling. Therefore, the negative slope of the V-wave is not that evident in these figures. The cycle then starts anew with atrial depolarization and increase in the LAP signal waveform corresponding to the A-wave.

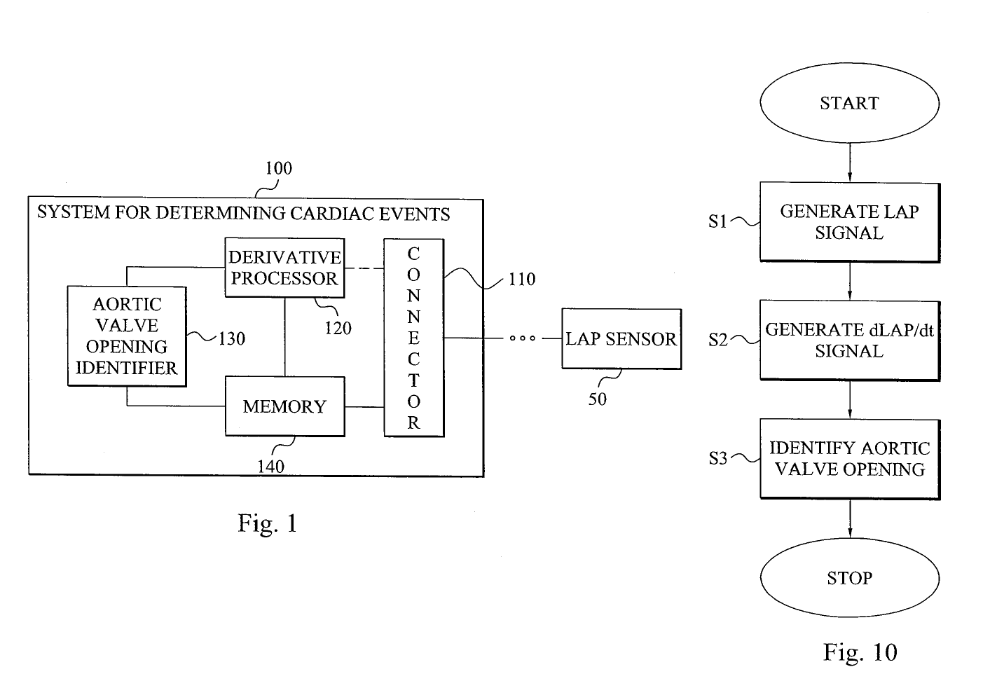

FIG. 1 is a schematic block diagram of an embodiment of a system 100 for determining cardiac events, and in particular characteristic heart valve related events. The system 100 comprises a connector 110 connectable to an implantable pressure sensor 50. This pressure sensor 50 is configured to generate a LAP signal representative of the left atrial pressure of a subject, preferably a mammalian subject and more preferably a human subject, during at least one, but preferably multiple, cardiac cycles.

The embodiments can be used in connection with any implantable sensor that is capable of recording a LAP signal or a signal that is at least representative of the LAP during at least one cardiac cycle. Such implantable sensors are known in the art. For instance, an implantable LAP sensor that can be used according to the embodiments is disclosed in U.S. Pat. No. 7,418,868 B1, the disclosure of which with regard to sensor design is incorporated herein by reference. Briefly, a pressure sensor module has a housing with a pressure sensor provided in the housing. The pressure sensor is electrically coupled to a plurality of electrical conductors extending into the housing through a feedthrough disposed within and hermetically sealing a first end of the housing. The housing defines a chamber between the electrical conductor feedthrough and a second end of the housing. This chamber contains a material in communication with the pressure sensor and is capable of transmitting pressure to the pressure sensor. The pressure sensor module can be provided on an endocardial lead, such as transseptal lead. The pressure sensor module is then advantageously arranged in connection with the distal end of the lead and comprises a longitudinally extending, electrically conductive housing electrically coupled to a terminal contact at the proximal end of the lead. The housing has a distal part projecting from a distal extremity of the lead body. The distal part of the housing can be used to function as a cardiac tissue electrical stimulation and/or sensing electrode. The module further comprises a pressure sensor disposed within the housing, where the sensor is electrically coupled to associated terminal contacts at the proximal lead end.

A further variant of a pressure sensor that can be used according to the embodiments is disclosed in U.S. Pat. No. 7,515,971 B1, the disclosure of which with regard to sensor design is incorporated herein by reference. The sensor is provided on a transseptal lead that accesses the left atrium through the septum between the right and left atrium. The lead includes a pressure sensor and a mounting mechanism on its distal end. This distal end of the lead is routed from a right side of the heart through the septum into the left atrium. A set of arms is coupled to the distal end to secure the lead.

The international application WO 2005/107583 A2 also discloses a pressure sensor that can be used according to the embodiments and the disclosure of which with regard to sensor design is incorporated herein by reference. A pressure sensor assembly comprises a sensing interface with at least one pressure sensing unit contacting a face of the sensing interface. A sensor housing is hermetically sealed to the pressure sensor.

An implantable LAP sensor that can be used according to the embodiments is employed in the so-called LAPTOP-HF trial. The LAP management system used in the trial comprises a small, pacemaker-sized, stand-alone implantable LAP monitoring device or a cardiac rhythm management device (cardiac resynchronization therapy (CRT) device or implantable cardioverter-defibrillator (ICD) device) with an integrated LAP monitoring feature. A lead or thin wire extending from the device towards the heart is equipped with an implantable LAP sensor.

The above mentioned LAP sensors should merely be seen as illustrative examples of implantable pressure sensors that can be used according to the invention and the embodiments are not limited thereto.

A derivative processor 120 of the system 100 is configured to process the LAP signal originating from the pressure sensor 50 and received by the connector 110. In particular, the derivative processor 120 generates or calculates a derivative LAP signal based on the LAP signal. This derivative LAP signal is representative of a first time derivative of the LAP signal.

The system 100 also comprises an aortic valve opening identifier 130 configured to process the derivative LAP signal in order to identify the time point of opening of an aortic valve of a heart in the subject. This time point of aortic valve opening coincides with a time point of a minimum in the derivative LAP signal following depolarization of the ventricles of the heart for a cardiac cycle. Thus, the time point for this minimum in the derivative LAP signal following ventricular depolarization, either intrinsic or stimulated, corresponds to the time point of aortic valve opening. The aortic valve opening identifier 130 thereby investigates the derivative LAP signal in order to identify the minimum and notes the time point of this minimum as the time point of aortic valve opening for the cardiac cycle.

The time point of aortic valve opening can be expressed in various ways. For instance, the derivative LAP signal from the derivative processor 120, and typically also the LAP signal from the pressure sensor 50, could be in the form of a stream or sequence of samples, where each signal sample has a value corresponding to the first time derivative of the LAP signal or to the LAP signal value at that particular time point. The aortic valve opening identifier 130 can then parse through the samples of the derivative LAP signal following the sample(s) coinciding with ventricular depolarization until the sample having the smallest value is found. The search for this minimum value could be limited to merely a portion of the samples, such as the samples following the ventricular depolarization until the end of the cardiac cycle. The search window can be limited even further, such as those samples following the ventricular depolarization up to the V-wave in the LAP signal waveform. Generally, this search window could extend up to about 0.5-0.6 s following ventricular depolarization.

The sample number of the identified sample having the minimum value could then be used as a representation of the time point of aortic valve opening. The sample number can also be converted to a true time value given the sampling frequency of the LAP derivative signal.

The time point of aortic valve opening is preferably defined relative a reference point in the cardiac cycle. Any easily identifiable characteristic in the cardiac cycle could be used as such reference point. Non-limiting examples include time point of atrial depolarization, either intrinsic sensed by the system 100 or stimulated by application of an atrial pacing pulse, the time point of ventricular depolarization, either intrinsic sensed by the system 100 or stimulated by application of a ventricular pacing pulse, or the time point of ventricular repolarization. These characteristic events can easily be identified in an electrogram signal recorded for the subject and/or be defined at the point of application of the atrial or ventricular pacing pulse. A typical example of a reference point is the start of a cardiac cycle. The cardiac cycle can then, as further disclosed herein, be defined, for instance, as the time interval between two consecutive R-waves of QRS complexes, i.e. an RR-interval. The start of a cardiac cycle would then coincide with the R-wave of the QRS complex.

The system 100 preferably also comprises a memory 140 that can store different signals and/or data derived from the signals. For instance, the memory 140 can store the LAP signal received by the connector 110 from the pressure sensor 50. The derivative processor 120 then calculates the derivative LAP signal from the data stored in the memory 140. Alternatively, the LAP signal samples are directly input to the derivative processor 120 from the connector 110.

The derivative LAP signal generated by the derivative processor 120 could be provided directly to the aortic valve opening identifier 130. Alternatively, or in addition it is stored in the memory 140, from which it can be accessed by the aortic valve opening identifier 130.

Correspondingly, information of the time point of aortic valve opening identified by the aortic valve opening identifier 130 can be stored in the memory 140 for later use as disclosed herein.

The time point of aortic valve opening identified by the aortic valve opening identifier 130 is preferably identified as corresponding to or coinciding with the time point of a global minimum in the derivative LAP signal during the cardiac cycle. Thus, the aortic valve opening does preferably not only coincide with a minimum in the derivative LAP signal following ventricular depolarization but this minimum is typically the global minimum in the derivative LAP signal for the cardiac cycle.

In such a case, the time point can easily be identified based on the sample number of the derivative LAP signal sample that has the smallest value during the cardiac cycle.

A cardiac cycle can, in similar to the reference point definition above, correspond to the cycle between any two consecutive easily identifiable characteristics. A preferred such cycle, is the RR-interval corresponding to the time point of the R-wave in the QRS complex for a preceding heart beat to the time point of the R-wave in the QRS complex of the following heart beat. Other examples include the PP-interval corresponding to the interval between P-waves in two consecutive heart beats, or less commonly the TT-interval, corresponding to the interval between T-waves in two consecutive heart beats.

Generally, there can be local differences in the LAP signal and thereby in the derivative LAP signal between different cardiac cycles due to various disturbing effects that do not relate to changes in LAP. For instance, slight movements of the pressure sensor or a change in subject position e.g. from supine position to sitting or standing could affect the LAP signal.

The effects of such disturbances on the LAP signal and the derivative LAP signal can be suppressed by calculating the average of the derivative LAP signal and/or the LAP signal over multiple cardiac cycles. For instance, an average LAP signal defined between RR-intervals can be calculated from the LAP signals from the pressure sensor 50 for multiple, preferably consecutive, cardiac cycles, such as 3-10 cardiac cycles. The derivative LAP signal is then generated by the derivative processor 120 from this average LAP signal, which also can be generated by the derivative processor 120. Alternatively, the derivative processor 120 can calculate an average derivative LAP signal from the derivative LAP signal for multiple, preferably consecutive, cardiac cycles, such as 3-10 cardiac cycles.

A further alternative is to determine a time point of aortic valve opening relative the reference point for multiple, preferably consecutive, cardiac cycles, such as 3-10 cardiac cycles. The time point of aortic valve opening could then be calculated as the average of the intervals between the reference point, typically ventricular depolarization, and the (global) minimum in the derivative LAP signal for each cardiac cycle.

The pressure sensor 50 can be used to record LAP signals continuously. Alternatively, the system 100 controls the pressure sensor 50 to periodically record the LAP signal at defined measurement intervals, such as once or a few times per day, per week or per month. A further alternative or addition is to record the LAP signal based on reception of a request. For instance, the physician can wirelessly send a request to the system 100 for recording LAP signals and identifying heart valve events as disclosed herein. In such a case, the system 100 activates the pressure sensor 50 to start a new measurement session based on the request. In the case of LAP measurements at defined measurement intervals or upon request, the system 100 preferably conducts the pressure measurements during a defined period of time, such as during a specified time period or during a specified amount of consecutive cardiac cycles. The length of such a measurement period can be defined by the physician and included in the request or otherwise programmed into the system 100.

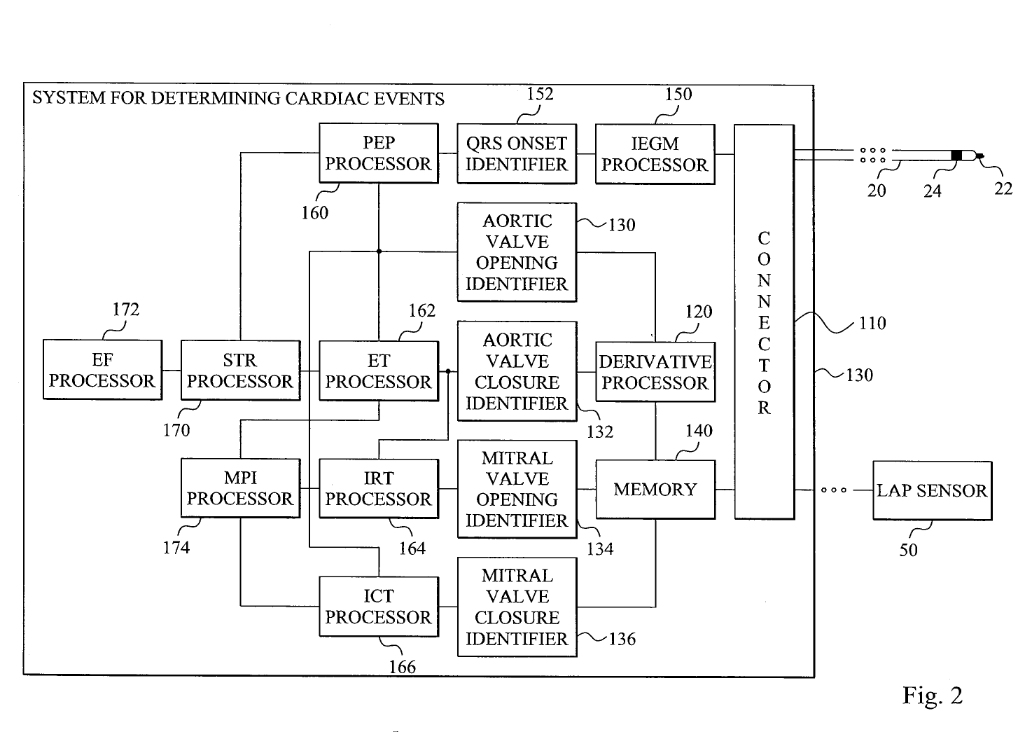

FIG. 2 is a schematic block diagram of another embodiment of a system 100 for determining cardiac events. This embodiment of the system 100 comprises the previously discussed connector 110 connectable to the implantable pressure sensor 50, the derivative processor 120, the aortic valve opening identifier 130 and the memory 140.

The connector 110 is preferably also configured to be connected to at least one cardiac lead 20 having at least one electrode 22, 24 provided at or in connection with the distal end of the cardiac lead 20. The opposite proximal end of the cardiac lead 20 is then designed with matching electrode terminal(s) that is (are) connected to the connector 110 to thereby achieve an electrical connection between the connector 110 and thereby the system 100 and the at least one electrode 22, 24 through at least one conductor running along the main body of the cardiac lead 20.

The electrode(s) 22, 24 of the cardiac lead 20 is (are), during operation in a subject body, provided in or in connection to a heart chamber and can be used by the system 100 for sensing electrical events, such as atrial and/or ventricular depolarization occurring during cardiac cycles in heart.

The electrical signals sensed by the cardiac lead 20 are preferably input to an electrogram processor 150, represented by an IEGM processor 150 in FIG. 2. The electrogram processor 150 is then configured to generate an electrogram signal, typically an IEGM signal, representative of the electric activity of the heart during cardiac cycles. The electrogram signal is advantageously processed by a QRS onset identifier 152 that identifies the time point of ventricular depolarization during a cardiac cycle. The ventricular depolarization is identified as the time point of onset of the QRS complex or as the R-wave portion of the QRS complex in the electrogram signal. As is well known in the art, such QRS complexes can easily be identified in electrogram signals, such as IEGM signals, as the main and most prominent feature in the electrogram signal during a cardiac cycle.

The time point of ventricular depolarization as determined based on the timing of the detected QRS complex in the electrogram signal can be used by the aortic valve opening identifier 130 in order to identify the start of the search window in which the minimum in the derivative LAP signal is to be found. In addition, the time point of ventricular depolarization can be used as a reference point as previously described so that the time of aortic valve opening can be defined relative the timing of ventricular depolarization.

The time period from the QRS complex as identified in the IEGM signal by the QRS onset identifier 152 to the opening of the aortic valve as identified by the aortic valve opening identifier 130 is of diagnostic value. The system 100 can therefore comprise a pre-ejection period (PEP) processor 160 that is configured to calculate a PEP parameter value for the subject based on the difference between the time point of onset of the QRS complex and the time point of aortic valve opening during a cardiac cycle, such as an average cardiac cycle.

The PEP parameter is of diagnostic value since a healthy heart should have a short pre-ejection period, whereas the PEP increases for diseased hearts and in particular if there are any problems with the ventricles.

The pre-ejection period consists of the electromechical coupling time followed by the mechanical muscular contraction plus the isovolumetric contraction that lasts until the opening of the aortic valve and ejection of blood into the aorta starts. As such PEP is inversely related to the efficiency of the heart. Contractility is one part of the efficiency and dyssynchrony another. The pre-ejection period includes the electromechanical delay for the electrical stimulus to activate the ventricular muscle and the isovolumetric contraction between mitral valve closure and aortic valve opening.

Many factors influence the value of the PEP.

Examples of factors that prolong the PEP are
Left ventricular heart failure
Ischemia
Decreased left ventricular (LV) dP/dt
LV conduction defects, typical left bundle branch block (LBBB)
Negative inotropic agents such as beta blockers
Decreased preload or venous return due to reduced blood volume from hemorrhage or standing upright
Atrial arrhythmias such as atrial fibrillation (AF) that impairs atrial contraction
Reduced ventricular filling time due to atrial tachycardia that reduces ventricular filling time
Ventricular diastolic failure by impaired relation or hypertrophy
Stenosis in mitral or tricuspid valve that reduces ventricular filling
Hypertension

Examples of factors that shorten the PEP are
Aortic valve disease with a compensated left ventricle
Diminished left ventricular isovolumic pressure
Positive inotropic agents such as dopamine
Rise in venous return
Acute drop in arterial afterload
Reduced heart rate

This means that the system 100 having a PEP processor 160 can be employed to monitor and detect various medical conditions as listed above by monitoring the PEP parameter value and detecting any (sudden) change, such as increase or decrease, in the PEP parameter value over time.

The monitoring performed based on the PEP parameter value typically involves storing PEP parameter values calculated by the PEP processor 160 at different time points in the memory 140. The PEP processor 160 can then calculate an average PEP parameter value based on the previously determined PEP parameter values, where the average could be a weighted average giving more weight to recently determined PEP parameter values as compared to previous values. Once the PEP processor 160 calculates a new PEP parameter value it compares it with the average PEP parameter value in the memory 140. If the new value differs significantly from the average value, i.e. more than a defined threshold value, this could be an indication of a medical condition as listed in the foregoing. If the new value does not differ significantly from the average value, the average PEP parameter value is preferably updated based on the new value.

The system 100, in an embodiment, not only determines the time point of aortic valve opening based on recorded LAP signals from the implantable pressure sensor 50. The system 100 could also process the derivative LAP signal in order to identify closure of the aortic valve. The system 100 then comprises an aortic valve closure identifier 132 configured to process the derivative LAP signal and identify a time point of closure of the aortic valve. The aortic valve closure is identified as coinciding with the time point of a maximum in the derivative LAP signal following aortic valve opening, i.e. the identified minimum in the derivative LAP signal. Thus, both the time point of opening and closure of the aortic valves can be determined by the system 100 using the same derivative LAP signal.

Once the system 100 has determined the time points of aortic valve opening and closure in a cardiac cycle, it can use these time points to calculate a diagnostic parameter. The system 100 then preferably comprises an ejection time (ET) processor 162 configured to calculate an ET parameter value. In more detail, the ET processor 162 calculates the ET parameter value based on the difference between the time point of aortic valve opening and the time point of aortic valve closure during a cardiac cycle, such as an average cardiac cycle.

The ET parameter is of diagnostic value since it reflects the health status of the heart. A healthy heart should generally have a long ejection time, which decreases for a dysfunctioning heart.

The ejection time is the time of ejection of blood from the left ventricle beginning with aortic valve opening and ending with aortic valve closure. The ejection time is increased during increase in arterial pressure, during increase in venous return and when significant aortic valve stenosis is present.

Left ventricular ejection time is decreased during left ventricular dysfunction not affecting LV outflow and during diminished venous return. The ejection time may decrease with both positive and negative inotropic agents.

A healthy ventricle has a short pre-ejection period and a long ejection time while a diseased ventricle has a long pre-ejection period and a short ejection time. Electromechanical systole (EMS) defined by the sum PEP+ET, shows very little change under such condition. Both the subcomponents of PEP (electromechanical delay and isovolumetric contraction) have been found to be prolonged in a failing left ventricle.

An embodiment of the system 100 having both the PEP processor 160 and the ET processor 162 could have a further unit that is able to determine a diagnostic parameter from the calculated PEP parameter value from the PEP processor 160 and the ET parameter value from the ET processor 162. This further diagnostic parameter is a systolic time ratio (STR) determined by an STR processor 170 of the system 100. The STR processor 170 is configured to calculate the STR parameter value for the subject based on a quotient between the PEP parameter value and the ET parameter value, preferably as STR=PEP/ET, wherein STR denotes the systolic time ratio parameter value, PEP denotes the pre-ejection period parameter value and ET denotes the ejection time parameter value.

The STR parameter is reflective of the myocardial contractility and can therefore be used in order to monitor the contractility status of the heart and/or detect any deteriorations in the contractility that can indicate reduced cardiac function and heart dysunction. Normal values for the STR parameter value for healthy subjects are typically 0.34±0.04 (European Heart Journal (2004) 25: 2185-2186). The STR parameter value increases in diseased ventricles indicating decrease of contractility or more dyssynchrony. The STR parameter was proposed by Weissler (Circulation (1968) 37: 149-59) to have a more heart rate independent index of heart performance. As PEP increases and ET decreases during heart failure those changes will be amplified when calculating the relation PEP/ET. As heart function deteriorates, the PEP increases and the left ventricular ejection time decreases resulting in an increasing STR.

Monitoring the STR could identify responses to therapy when STR decreases while increases in STR could signal risk of heart decompensation.

The STR parameter value calculated by the STR processor 170 can further be used by the system 100 in order to calculate yet another diagnostic parameter. In such an embodiment, the system 100 comprises an ejection fraction (EF) processor 172. The EF processor 172 is configured to calculate an EF parameter value for the subject based on the STR parameter value from the STR processor 170. In a particular embodiment, the EF processor 172 calculates the EF parameter value as 0.84−0.64×STR. This relationship between the STR parameter and the EF parameter is generally referred to as the Capan method (Critical Care Medicine (1987) 15: 402). Another relationship between the STR parameter and the EF parameter has been referred to as the Weissler method (Circulation (1970) 42: 455-462) and defines EF=1.125−1.25×STR. Other models for determining the ejection fraction from the STR parameter have been reported in Huml and Beus-Huml, “A new noninvasive method for the left ventricular dimension and ejection function assessment by two algorithms”, Institute for Health Protection, Sarajevo, 1999: EF=1.365e^{-2.35×STR}.

The EF parameter value decreases for a dysfunctioned heart as compared to a healthy heart.

Ejection fraction is a measure of myocardial contractility reflecting the effectiveness of ventricular ejection. It is calculated by dividing the stroke volume by the end diastolic volume, and it is reported as a percentage. A normal ejection fraction is greater than 55%, while considered poor when less than 35%. However EF is also sensitive to changes in afterload.

In an embodiment the system 100 not only identifies the time point of aortic valve activities but also of mitral valve activities. The system 100 could then comprise a mitral valve opening identifier 134 configured to process the LAP signal originating from the pressure sensor 50. The mitral valve opening identifier 134 identifies a time point of opening of the mitral valve of the heart in the subject to coincide with a time point of a maximum of the so-called V-wave in the LAP signal for a cardiac cycle, such as average LAP signal.

The time points of mitral valve opening and aortic valve closure as determined in an embodiment of the system 100 can be used to calculate a further diagnostic parameter in terms of isovolumetric relaxation time (IRT). In this embodiment the system 100 comprises an IRT processor 164 configured to calculate an IRT parameter value for the subject based on a difference between the time point of aortic valve closure as determined by the aortic valve closure identifier 132 and the time point of mitral valve opening as determined by the mitral valve opening identifier 134.

Myocardial relaxation is an active and energy consuming phase in the cardiac cycle. Ischemia and heart failure prolong this interval. Thus, an increase in the IRT parameter value can be used to detect heart failure and ischemia for a subject.

In an embodiment, the system 100 comprises a mitral valve closure identifier 136 configured to process the LAP signal in order to identify a time point of closure of the mitral vale of the heart. The mitral valve closure identifier 136 identifies the mitral valve closure as coinciding in time with the time point of a maximum of an A-wave in the LAP signal for a cardiac cycle, such as an average cardiac cycle.

The time point of mitral valve closure and the time point of aortic valve opening can be used by the system to calculate a further diagnostic parameter in terms of an isovolumetric contraction time (ICT). The system 100 then comprises an ICT processor 166 configured to calculate an ICT parameter value for the subject based on a difference between the time point of mitral valve closure as determined by the mitral valve closure identifier 136 and the time point of aortic valve opening as determined by the aortic valve opening identifier 130.

Ischemia and heart failure prolong the ICT and the ICT parameter can therefore be used to detect any ischemic or heart failure event for the subject.

In an embodiment the system 100 comprises a myocardial performance index (MPI) processor 174 configured to calculate a MPI parameter value for the subject. The MPI processor 174 then calculates this MPI parameter value (MPI) based on a quotient between the sum of the IRT parameter value (IRT) calculated by the IRT processor 164 and the ICT parameter value (ICT) calculated by the ICT processor 166 and the ET parameter value (ET) calculated by the ET processor 162, preferably as

$$MPI = \frac{IRT + ICT}{ET} .$$

MPI can be used to characterize both systolic and diastolic heart function. MPI is a sensitive indicator for symptomatic heart failure and for left ventricular dysfunction in patients suffering from cardiomyopathy and coronary artery disease.

The MPI parameter is also denoted as TEI index in the art. In a failing heart, the contraction and relaxation become slower, which is seen as an increase in the MPI parameter with deterioration of cardiac function.

The memory 140 of the system 100 can be used to store the “raw” LAP signal and the derivative LAP signal, optionally as respective average signals. The memory 140 can in addition or alternatively store the time points of heart valve events determined by the aortic valve opening identifier 130, the aortic valve closure identifier 132, the mitral valve opening identifier 134 and/or the mitral closure identifier 136. The memory 140 can also or instead be used to store the diagnostic parameter values obtained from the PEP processor 160, the ET processor 162, the IRT processor 164, the ICT processor 166, the STR processor 170, the EF processor 172 and/or the MPI processor 174.

The diagnostic parameters could be provided to a display (not illustrated) of or connected, such as by a wired or wireless connection, with the system 100. A physician can review the determined diagnostic parameters on the screen in order to see any changes in parameter values over time that could indicate a deterioration of the cardiac status or the effect of medication as mentioned above.

The determined diagnostic parameters could also be used to adjust any therapy applied to the subject by the system 100, for instance by an implantable medical device (IMD) in the form of a pacemaker or implantable cardioverter-defibrillator (ICD) to be further described herein. In such a case, the pacing scheme employed by the IMD to treat the patient can be optimized by monitoring the effect of the pacing treatment on at least one of the diagnostic parameters. For instance, cardiac resynchronization therapy (CRT) settings, including for instance atrioventricular (AV) intervals and/or interventricular (VV) intervals, could be adjusted and set based on the values of at least one of the parameters.

The diagnostic parameters determined by the system 100 can also be used to trigger alarms if any of the parameters indicate a sudden deterioration of the subject's cardiac function. The alarm could be a visible alarm, an audio alarm or a tactile alarm that alerts the subject of the potentially life-threatening situation and urges him/her to seek medical assistance.

The system 100 as illustrated in FIG. 2 includes all the functions necessary in order to monitor all heart events relating to the aortic and mitral valves and to calculate all of the above-mentioned diagnostic parameters. The embodiments are, however, not limited thereto. In other embodiments only a single diagnostic parameter or a subset of the diagnostic parameters are needed. In such a case, the system 100 only needs to comprise the identifier(s) and processor(s) required in order to determine the time point(s) of the relevant heart event(s) and calculate the desired diagnostic parameter(s). Hence, modifications of the system 100 in FIG. 2 can be obtained and are within the scope of the embodiments by omitting one or some of the identifiers and processors illustrated in FIG. 2.

FIG. 5 is a diagram plotting the LAP signal (unbroken line) and the derivative LAP signal (broken line) recorded for a human subject. The characteristic features of the LAP signal, i.e. the A-wave and the V-wave are indicated in the figure together with heart valve events that can be detected by processing the LAP signal and the derivative LAP signal as disclosed herein.

In summary, the mitral valve closure is obtained as the point in time at which the A-wave has reached its maximum. The mitral valve opening is identified by detecting the point in time where the maximum of the V-wave has occurred. The minimum value of the derivative LAP signal corresponding to the largest negative rate of change of the LAP signal following the point identified as mitral valve closing corresponds to the opening of the aortic valve. Similarly, the maximum value of the derivative LAP signal corresponding to the largest positive rate of change of the LAP signal shortly before the point identified as the mitral valve opening is intimately related to the aortic valve closure.

The system 100 of the embodiments can therefore be used to provide interesting physiological and diagnostic parameters, including PEP, ET, ICT, IRT, STR, MPI and EF, without having to use echocardiography or other time and resource consuming techniques. As indicated above, the parameters derivable from the timing of heart valve events as determined from processing of the recorded LAP signal can be used to detect or monitor the progression of heart failure, the effects of medication and/or warn for exacerbations.

FIG. 6 schematically illustrates the relationship between PEP parameter values calculated according to the embodiments based on the LAP signal recorded by an implantable pressure sensor with PEP parameter values obtained from echocardiography measurements. The high linearity between the two measurement methods indicate that processing of LAP signals according to the embodiments can be used instead of traditional echocardiography measurements in order to derive valuable diagnostic parameters.

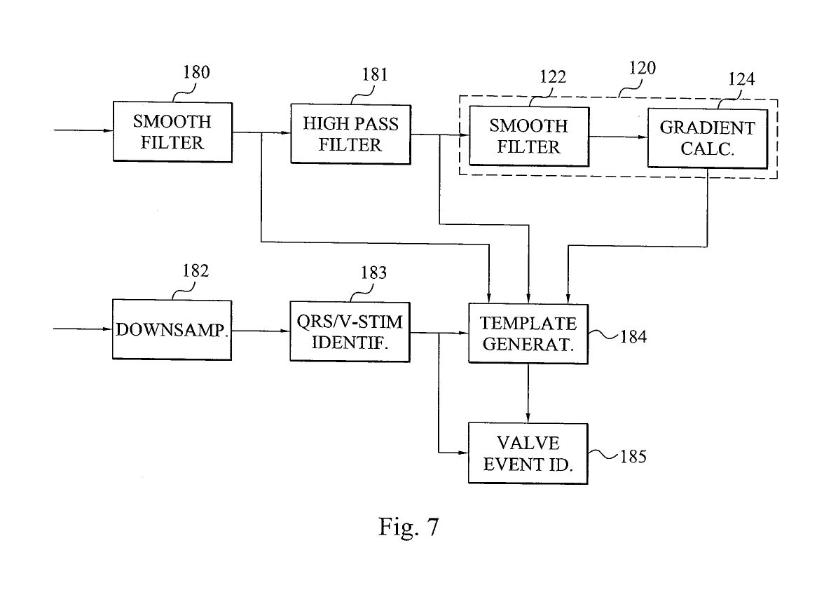

FIG. 7 schematically illustrates an embodiment of the co-processing of the LAP signal and the IEGM signal in order to identify the relevant heart valve events. A smoothing filter 180 can be used to filter the LAP signal in order to suppress high frequency noise in the LAP signal. The filtered LAP signal is then input to a high pass filter 181 configured to filter the LAP signal further to suppress the respiratory variations in the LAP signal. The derivative processor 120 can be implemented as a smoothing filter 122 and a gradient calculator 124. The smoothing filter 122 filters the LAP signal output from the high pass filter 181 to form a smoothed LAP signal. This smoothed LAP signal is input to the gradient calculator 124 to calculate the derivative LAP signal.

In a particular embodiment the LAP signal input to the smoothing filter 180 is sampled at 200 Hz and is lightly smoothed at 30 Hz to remove high frequency noise. The smoothing filter 180 could then be a 5 point Gaussian filter or a 4^{th} order low pass filter (30 Hz). The high pass filter 181 can be implemented as a 2^{nd} order high pass filter (0.75 Hz) to remove most of the respiration variation in the LAP signal. The smoothing filter 122 of the derivative processor could be a 1^{st} order low pass filter (5-10 Hz) to provide further smoothing at the interval from 5 Hz to 10 Hz, preferably at 5 Hz. In this embodiment, the derivative LAP signal will be highly smoothed. However, according to the embodiments the derivative LAP signal is employed in order to identify time points of heart valve events and not to define an absolute amplitude of the derivative LAP signal.

The gradient calculator 124 can be implemented to calculate derivative LAP signal samples based on the difference between two consecutive LAP signal samples divided by the sampling frequency, i.e. as

$${( \frac{d LAP}{d t} )}_{i} = \frac{{( LAP )}_{i} - {( LAP )}_{i - 1}}{1 / 200} ,$$

where

$${( \frac{d LAP}{d t} )}_{i}$$

denotes the derivative value of sample i in the derivative LAP signal and (LAP)ᵢ denotes the LAP value of sample i in the LAP signal.

In an embodiment, the IEGM signal is sampled at 400 Hz. The IEGM signal is, in an embodiment, time aligned with the LAP signal by downsampling to 200 Hz at a downsampling unit 182. A following unit 183 corresponds to the QRS onset identifier of FIG. 2 and is employed to identify electrical events in the form of QRS wave and/or ventricular stimulation (V-stim). In an embodiment, cardiac cycles, i.e. RR-intervals in the downsampled IEGM signal, containing ventricular extrasystoles (VES) are preferably removed by the unit 183.

A template generator 184 is preferably connected to the outputs of the smoothing filter 180, the high pass filter 181, the gradient calculator 124 and the unit 183 to create signal templates using signal averaging of the included RR intervals in the input signals, i.e. filtered LAP signal from the smoothing filter 180, high pass filtered LAP signal from the high pass filter 181, derivative LAP signal from the gradient calculator 124 and IEGM signal from the unit 183. In other embodiments, other signal processing techniques besides template creation can be used including, for instance, calculating the median value for a feature over several RR-intervals.

In an embodiment, the template generator 184 first calculates an average LAP value as the average of the filtered LAP signal template created from the filtered LAP signal from the smoothing filter 180. The LAP signal template is then calculated as the sum of the high pass filtered LAP signal template created from the high pass filtered LAP signal from the high pass filter 182 and the average LAP value.

The LAP signal template, a derivative LAP signal template created from the derivative LAP signal from the gradient calculator 124 and an IEGM signal template created from the IEGM signal from the unit 183 are output from the template generator 184 and to a valve event identifier 185 that corresponds to the identifiers of FIG. 2.

In such a case, the mitral valve closure identifying function of the valve event identifier 185 can identify the time point of mitral valve closure corresponding to the A-wave maximum of the LAP signal template in a search window extending from 0 ms to 250 ms of the RR-interval, e.g. from the onset or maximum of the QRS complex in the IEGM template. The mitral valve opening identifying function of the valve event identifier 185 preferably identifies the time point of mitral valve opening corresponding to the V-wave maximum of the LAP signal template in a search window extending from 300 ms to 80% of the length of the RR-interval. Correspondingly, the aortic valve opening identifying function of the valve event identifier 185 identifies the time point of aortic valve opening corresponding to a (global) minimum in the derivative LAP signal template in a search window extending from 0 ms to 300 ms of the RR-interval. Finally, the aortic valve closure identifying function of the valve event identifier 185 preferably identifies the aortic valve closure to correspond to a maximum in the derivative LAP signal template in a search window extending from 200 ms to 80% of the RR-interval.

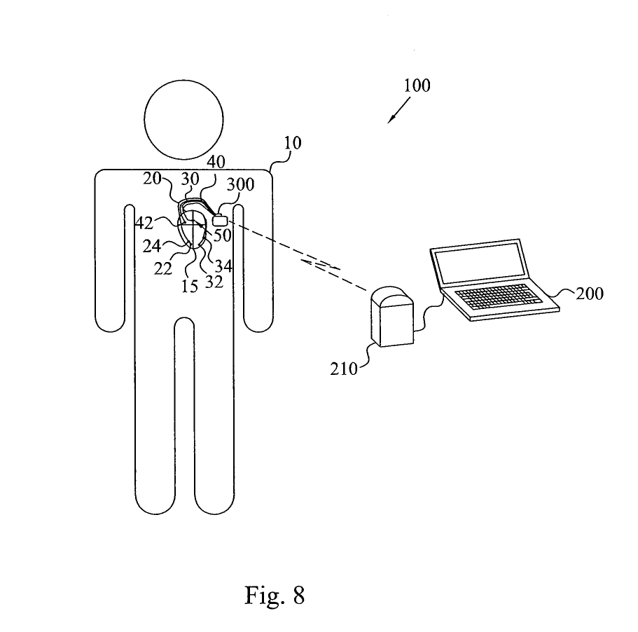

FIG. 8 is a schematic overview of a subject 10 and a system 100 determining cardiac events according to the embodiments. The system 100 comprises an implantable medical device (IMD) 300, which could be in the form of a pacemaker, cardiac resynchronization device or an ICD having at least one cardiac lead 20, 30, 40 equipped with an implantable pressure sensor 50 provided in connection with the subject's heart 15 to measure the left atrial pressure. In the figure, the IMD 300 has a connected right ventricular lead 20 with electrodes 22, 24 provided in the right ventricle of the heart 15 in order to enable right ventricular sensing and/or pacing. In an embodiment, the IMD 300 is also or alternatively connected to a left ventricular lead 30 having electrodes 32, 34 provided in connection with the left ventricle in order to enable left ventricular sensing and/or pacing. The left ventricular lead 30 is generally provided epicardially in the coronary system of the heart 15. A right atrial lead 40 has an electrode 42 positioned in the right atrium of the heart 15 to enable atrial sensing and/or pacing.

The pressure sensor 50 as illustrated in FIG. 8 is positioned on a separate lead or wire entering the right atrium in the heart and positioning the pressure sensor 50 in the septum between the right and left atria to thereby place the pressure-sensitive part of the pressure sensor 50 in the left atrium. The embodiments are, however, not limited to this particular equipment for LAP measurements. For instance, the pressure sensor 50 could be positioned on the right atrial lead 40 which then could have both pacing/sensing electrode(s) 42 and the pressure sensor 50.

The IMD 300 can advantageously wirelessly communicate with a non-implantable data processing device 200, such as a programmer of physician's workstation. The data processing device 200 then comprises or is connected to a transceiver 210 that performs the wireless communication with a corresponding transceiver in the IMD 300.

In an embodiment, the system 100 comprises both the IMD 300 and the data processing device 200. In such a case, the IMD 300 comprises the connector 110 of the system 100 to which the pressure sensor 50 is connected, see FIG. 2. In addition, the IMD 300 preferably comprises the IEGM processor 150. In such a case, the processing of the LAP signal and the identification of heart valve events and calculation of diagnostic parameters can be performed by the data processing unit 200. The data processing unit 200 thereby comprises the identifiers 130-136, 152 and the processors 160-174 of FIG. 2. Generally, the data processing device 200 has enhanced data processing capability as compared to the IMD 300, which inherently is limited in size to be implantable and is powered by a battery and therefore has limited processing power.

The IMD 300 then gathers the LAP signal from the implantable sensor 50 and preferably the IEGM signal from the IEGM processor 150. Data from these signals is then wirelessly transmitted to the transceiver 210 and the data processing device 200, where the heart valve events are identified and the diagnostic parameters can be calculated. Information of the timing of the heart valve events and/or the diagnostic parameters can then be displayed on a screen of or connected to the data processing device 200.

In an alternative embodiment the IMD 300 additionally comprises the identifiers 130-136 and preferably the QRS onset identifier 152. In such a case, the IMD 300 will determine the timings of the heart valve events and information thereof is transmitted to the data processing device 200. The data processing device then comprises the processors 160-174 that are employed to calculate the diagnostic parameters from the received information of the timings of the heart valve events.

It is in fact possible to have any distribution of identifiers and processors as illustrated in FIG. 2 between the IMD 300 and the data processing device 200.

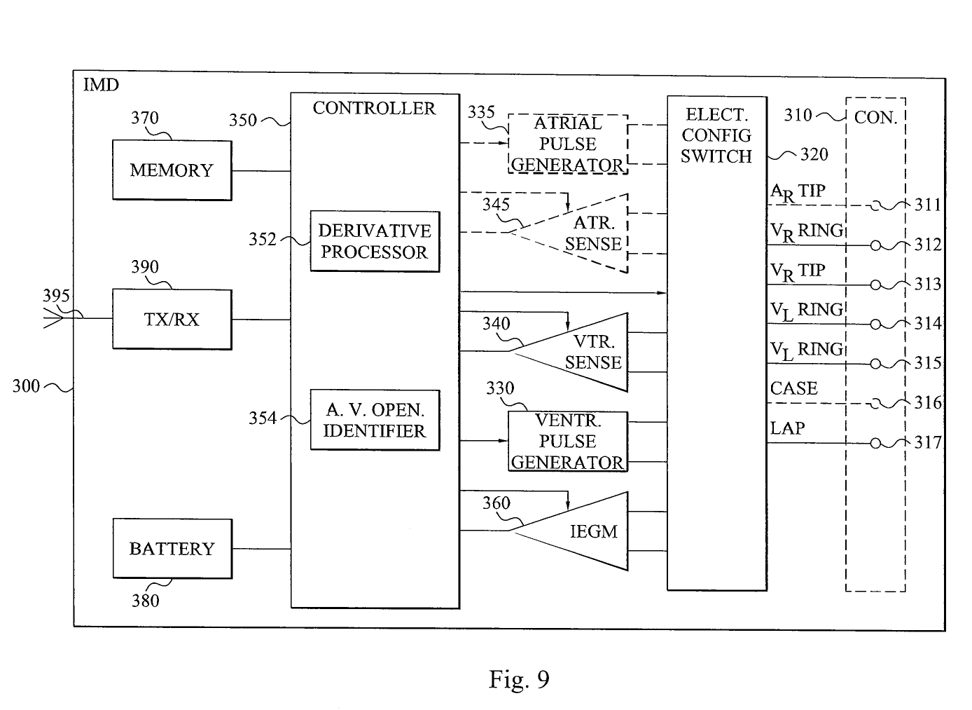

The above described embodiments therefore relate to a distributed implementation of the system 100 between the IMD 300 and the data processing device 200. In an alternative embodiment, the system 100 is implemented in the IMD 300. Thus, the IMD 300 then comprises the connector, the derivative processor and the aortic valve opening identifier and preferably any of the optional other identifiers and the optional processors illustrated in FIG. 2. Such an IMD-based implementation of the system 100 is illustrated in FIG. 9.

FIG. 9 is a schematic block diagram of an IMD 300 according to an embodiment. FIG. 9 is a simplified block diagram depicting various components of the IMD 300. While a particular multi-chamber device is shown, it is to be appreciated and understood that this is done merely for illustrative purposes. Thus, the techniques and methods described below can be implemented in connection with other suitably configured IMDs. Accordingly, the person skilled in the art can readily duplicate, eliminate, or disable the appropriate circuitry in any desired combination.

The IMD 300 comprises a housing, often denoted as can or case in the art. The housing can act as return electrode (case electrode) for unipolar leads, which is well known in the art. The IMD 300 also comprises a connector 310 having, in this embodiment, a plurality of terminals 311-317. The terminals 311-315 are configured to be connected to matching electrode terminals of implantable medical leads connectable to the IMD 300 and the connector 310. FIG. 9 illustrates an embodiment with terminals 311-315 that corresponds to the lead embodiment as illustrated in FIG. 8, i.e. having a right atrial lead 40 with a tip electrode 42, a right ventricular lead 20 with a tip electrode 22 and a ring electrode 24 and a left ventricular lead 30 with two ring electrodes 32, 34. If other lead embodiments are employed, the terminals of the connector 310 are appropriately modified to match the electrode terminals of the at least one connectable implantable medical lead.

The connector 310 is optionally connected to at least one case electrode and thereby has a matching terminal 316 for this case electrode.

According to the embodiments, the IMD 300 and the connector 310 are connectable to the implantable pressure sensor and thereby have one or more terminals 317 configured to receive the LAP representing signal from the pressure sensor. The pressure sensor can be provided on a separate lead or wire or could be attached to or forming part of any of the connected cardiac leads.

If the IMD 300 is connectable to an atrial lead, the IMD 300 comprises an atrial pulse generator 335 generating pacing pulses for delivery by the atrial lead(s) preferably through an electronic configuration switch 320. The IMD 300 preferably also comprises a ventricular pulse generator 330 that generates pacing pulses for delivery by the ventricular lead(s) to the left and/or right ventricle.

It is understood that in order to provide stimulation therapy in different heart chambers, the atrial and ventricular pulse generators 330, 335 may include dedicated, independent pulse generators, multiplexed pulse generators, or shared pulse generators. The pulse generators 330, 335 are controlled by a controller 350 via appropriate control signals, respectively, to trigger or inhibit the stimulating pulses.

The controller 350 of the IMD 300 is preferably in the form of a programmable microcontroller 350 that controls the operation of the IMD 300. The controller 350 typically includes a microprocessor, or equivalent control circuitry, designed specifically for controlling the delivery of pacing therapy, and may further include RAM or ROM memory, logic and timing circuitry, state machine circuitry, and I/O circuitry. Typically, the controller 350 is configured to process or monitor input signal as controlled by a program code stored in a designated memory block. The type of controller 350 is not critical to the described implementations. In clear contrast, any suitable controller may be used that carries out the functions described herein. The use of microprocessor-based control circuits for performing timing and data analysis functions are well known in the art.

The optional electronic configuration switch 320 includes a plurality of switches for connecting the desired terminals 311-317 to the appropriate I/O circuits, thereby providing complete electrode programmability. Accordingly, the electronic configuration switch 320, in response to a control signal from the controller 350, determines the polarity of the stimulating pulses (e.g. unipolar, bipolar, combipolar, etc.) by selectively closing the appropriate combination of switches (not shown) as is known in the art.

An optional atrial sensing circuit or detector 345 and a ventricular sensing circuit or detector 340 are also selectively coupled to the atrial lead(s) and the ventricular lead(s) through the switch 320 for detecting the presence of cardiac activity in the heart chambers. Accordingly, the ventricular and atrial sensing circuits 340, 345 may include dedicated sense amplifiers, multiplexed amplifiers, or shared amplifiers. The switch 320 determines the “sensing polarity” of the cardiac signal by selectively closing the appropriate switches, as is also known in the art. In this way, the clinician may program the sensing polarity independent of the stimulation polarity. The sensing circuits are optionally capable of obtaining information indicative of tissue capture.

Each sensing circuit 340, 345 preferably employs one or more low power, precision amplifiers with programmable gain and/or automatic gain control, band-pass filtering, and a threshold detection circuit, as known in the art, to selectively sense the cardiac signal of interest.

The outputs of the ventricular and atrial sensing circuits 340, 345 are connected to the controller 350, which, in turn, is able to trigger or inhibit the ventricular and atrial pulse generators 330, 335, respectively, in a demand fashion in response to the absence or presence of cardiac activity in the appropriate chambers of the heart.

Furthermore, the controller 350 is also typically capable of analyzing information output from the sensing circuits 340, 345 and/or an IEGM processor 360 to determine or detect whether and to what degree tissue capture has occurred and to program a pulse, or pulse sequence, in response to such determinations. The sensing circuits 340, 345, in turn, receive control signals over signal lines from the controller 350 for purposes of controlling the gain, threshold, polarization charge removal circuitry, and the timing of any blocking circuitry coupled to the inputs of the sensing circuits 340, 345 as is known in the art.

Cardiac signals are applied to inputs of the IEGM processor 360 connected to the lead connector 310. The IEGM processor 360 is preferably in the form of an analog-to-digital (A/D) data acquisition unit configured to acquire IEGM signals, convert the raw analog data into a digital signal, and store the digital signals for later processing and/or transmission to a programmer by a transmitter or transceiver 390. The IEGM processor 360 is coupled to the atrial lead and/or the ventricular lead and optionally the case electrode through the switch 320 to sample cardiac signals across any pair of desired electrodes.

Advantageously, the operating parameters of the IMD 300 may be non-invasively programmed into a memory 370 through a receiver or transceiver 390 in communication via a communication link with the previously described communication unit of the programmer. The controller 350 activates the transceiver 390 with a control signal. The transceiver 390 can alternatively be implemented as a dedicated receiver and a dedicated transmitter connected to separate antennas or a common antenna, preferably a radio frequency (RF) antenna 395.

The IMD 300 additionally includes a battery 380 that provides operating power to all of the circuits shown in FIG. 9.

In this embodiment, the derivative processor 352 and the aortic valve opening identifier 354 are implemented as being run by the controller 350. The IMD 300 may additionally comprise any of the aortic valve closure identifier, the mitral valve opening identifier and the mitral valve closure identifier. In such a case, also these identifiers can be run by the controller 350. Furthermore, any of the previously described QRS onset identifier, PEP processor, ET processor, STR processor, EF processor, IRT processor, ICT processor and the MPI processor can be implemented in the IMD 300, preferably run by the controller 350.

These units can then be implemented as a computer program product stored in the memory 370 and loaded and run on a general purpose or specially adapted computer, processor or microprocessor, represented by the controller 350 in FIG. 9. The software includes computer program code elements or software code portions effectuating the operation of the units. The program may be stored in whole or part, on or in one or more suitable computer readable media or data storage means that can be provided in an IMD 300.

In an alternative embodiment, the units are implemented as hardware units either forming part of the controller 350 or provided elsewhere in the IMD 300.

The diagnostic parameter(s) calculated by the IMD 300 can, as mentioned in the foregoing, be used by the controller 350 in order to control the operation of the IMD 300. For instance, the diagnostic parameter(s) can be used to select optimal or suitable CRT settings, such as AV and/or VV delays. In such a case, the controller 350 tests various CRT settings by changing the time intervals between causing the atrial pulse generator 335 and/or the ventricular pulse generator 330 to generate and apply pacing pulses according to various candidate AV and/or VV delays. The IMD 300 then determines at least one diagnostic parameter value for each tested candidate CRT setting and then selects the CRT setting that resulted in the most optimal hemodynamic response as assessed by the values of the diagnostic parameter(s).

Correspondingly, if any of the monitored diagnostic parameters indicate a potentially life-threatening cardiac condition, the controller 350 could initiate combating actions, such as selecting a particular pacing scheme or the application of a defibrillation shock by controlling the ventricular and atrial pulse generator 330, 335 to generate such a defibrillation shock.

In addition or alternatively, the controller 350 can generate an alarm or emergency message that is transmitted by the transceiver 390 to a non-implantable device, such as the previously discussed data processing device, in order to inform the subject and/or his/her physician of the deterioration in cardiac status as detected by monitoring at least one of the diagnostic parameters that are derivable from the LAP signal.

FIG. 10 is a flow diagram illustrating a method for identifying cardiac valve events according to an embodiment. The method starts in step S1, where a LAP signal representative of the left atrial pressure of a subject during at least one cardiac cycle is generated or recorded. A derivative LAP signal is calculated in step S2 based on the LAP signal from step S1. The derivative LAP signal then represents the first time derivative of the LAP signal. A next step S3 identifies a time point of opening of the aortic valve to coincide with the time point of a minimum in the derivative LAP signal following ventricular depolarization during a(n) (average) cardiac cycle. In a particular embodiment of step S3, the aortic valve opening is identified as occurring at the time point of global minimum in the derivative LAP signal during the cardiac cycle.

The method may additionally comprise processing of the derivative LAP signal and/or the LAP signal in order to identify the time point of aortic valve closure, mitral valve opening and/or mitral valve closure as disclosed herein.

The method also advantageously comprises generating an electrogram signal representative of the electric activity of the heart during at least one cardiac cycle. Depolarization of the ventricles in the to (average) cardiac cycle can then be identified to coincide with a time point of onset of the QRS complex in the electrogram signal. If ventricular stimulation is applied to the patient, the time point of ventricular depolarization preferably corresponds to the point in time at which the ventricular pacing pulse is applied to the subject's heart.

The method may additionally comprise determining any of the previously discussed diagnostic parameters, such as the PEP parameter, ET parameter, STR parameter, EF parameter, IRT parameter, ICT parameter and/or MPI parameter.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1. A system for determining cardiac events, said system comprising:
a connector connectable to an implantable pressure sensor configured to generate a left atrial pressure, LAP, signal representative of a left atrial pressure of a subject during at least one cardiac cycle;
a derivative processor configured to process said LAP signal and generate a derivative LAP signal representative of a first time derivative of said LAP signal; and
an aortic valve opening identifier configured to process said derivative LAP signal and identify a time point of opening of an aortic valve of a heart in said subject to coincide with a time point of a minimum in said derivative LAP signal following depolarization of ventricles of said heart for a cardiac cycle.

2. The system according to claim 1, wherein said aortic valve opening identifier is configured to identify said time point of opening of said aortic valve to coincide with a time point of a global minimum in said derivative LAP signal during said cardiac cycle.

3. The system according to claim 1, further comprising:
an electrogram processor configured to generate an electrogram signal representative of electric activity of said heart during said at least one cardiac cycle; and
a QRS onset identifier configured to process said electrogram signal and identify said depolarization of ventricles to coincide with a time point of onset of a QRS complex for said cardiac cycle.

4. The system according to claim 1, wherein said connector is connectable to at least one ventricular lead having at least one electrode configured to be arranged in or in connection with a ventricle of said heart, said system further comprising a ventricular pulse generator configured to generate pacing pulses applied to said ventricle using said at least one ventricular lead to trigger said depolarization of said ventricles.

5. The system according to claim 1, further comprising:
an electrogram processor configured to generate an electrogram signal representative of electric activity of said heart during said at least one cardiac cycle;
a QRS onset identifier configured to process said electrogram signal and identify a time point of onset of a QRS complex for said cardiac cycle; and
a pre-ejection period processor configured to calculate a pre-ejection period parameter value for said subject based on a difference between said time point of onset of said QRS complex and said time point of opening of said aortic valve.

6. The system according to claim 5, further comprising:
an aortic valve closure identifier configured to process said derivative LAP signal and identify a time point of closure of said aortic valve to coincide with a time point of a maximum in said derivative LAP signal following said minimum in said derivative LAP signal for said cardiac cycle; and
an ejection time processor configured to calculate an ejection time parameter value for said subject based on a difference between said time point of opening of said aortic valve and said time point of closure of said aortic valve.

7. The system according to claim 6, further comprising a systolic time ratio processor configured to calculate a systolic time ratio parameter value for said subject based on a quotient between said pre-ejection period parameter value and said ejection time parameter value.

8. The system according to claim 7, further comprising an ejection fraction processor configured to calculate an ejection fraction parameter value for said subject as 0.84−0.64×STR, wherein STR denotes said systolic time ratio parameter value.

9. The system according to claim 1, further comprising:
an aortic valve closure identifier configured to process said derivative LAP signal and identify a time point of closure of said aortic valve to coincide with a time point of a maximum in said derivative LAP signal following said minimum in said derivative LAP signal for said cardiac cycle;
a mitral valve opening identifier configured to process said LAP signal and identify a time point of opening of a mitral valve of said heart in said subject to coincide with a time point of a maximum of a V-wave in said LAP signal for said cardiac cycle; and
an isovolumetric relaxation time processor configured to calculate an isovolumetric relaxation time parameter value for said subject based on a difference between said time point of closure of said aortic valve and said time point of opening of said mitral valve.

10. The system according to claim 1, further comprising:
a mitral valve closure identifier configured to process said LAP signal and identify a time point of closure of a mitral valve of said heart in said subject to coincide with a time point of a maximum of an A-wave in said LAP signal for said cardiac cycle; and
an isovolumetric contraction time processor configured to calculate an isovolumetric contraction time parameter value for said subject based on a difference between said time point of closure of said mitral valve and said time point of opening of said aortic valve.

11. The system according to claim 6, further comprising a myocardial performance index processor configured to calculate a myocardial performance index parameter value (MPI) for said subject based on a quotient between a sum of said isovolumetric relaxation time parameter value (IRT) and said isovolumetric contraction time parameter value (ICT) and said ejection time parameter value (ET),
$$MPI = \frac{IRT + ICT}{ET} .$$

12. The system according to claim 1, wherein said connector, said derivative processor and said aortic valve opening identifier are implemented in an implantable medical device and wherein said connector is connectable to at least one cardiac lead having at least one electrode configured to sense electrical activity of said heart.

13. The system according to claim 1, wherein said connector is implemented in an implantable medical device having a transceiver configured to transmit information of said LAP signal to a transceiver of a non-implanted data processing device comprising said derivative processor and said aortic valve opening identifier.

14. A method for identifying cardiac valve events, said method comprising:
generating a left atrial pressure, LAP, signal representative of a left atrial pressure of a subject during at least one cardiac cycle;
calculating, based on said LAP signal, a derivative LAP signal representative of a first time derivative of said LAP signal; and
identifying a time point of opening of an aortic valve of a heart in said subject to coincide with a time point of a minimum in said derivative LAP signal following depolarization of ventricles of said heart for a cardiac cycle.

15. The method according to claim 14, wherein identifying said time point of opening of said aortic valve comprises identifying said time point of opening of said aortic valve to coincide with a time point of a global minimum in said derivative LAP signal during said cardiac cycle.

16. The method according to claim 14, further comprising:
generating an electrogram signal representative of electric activity of said heart during said at least one cardiac cycle; and
identifying, by processing said electrogram signal, said depolarization of ventricles to coincide with a time point of onset of a QRS complex for said cardiac cycle.

17. The method according to claim 14, further comprising generating pacing pulses applied to a ventricle of said heart by at least one ventricular lead having at least one electrode configured to be arranged in or in connection with said ventricle to trigger said depolarization of said ventricles.

18. The method according to claim 14, further comprising:
generating an electrogram signal representative of electric activity of said heart during said at least one cardiac cycle;
identifying, by processing said electrogram signal, a time point of onset of a QRS complex for said cardiac cycle; and
calculating a pre-ejection period parameter value for said subject based on a difference between said time point of onset of said QRS complex and said time point of opening of said aortic valve.

19. The method according to claim 18, further comprising:
identifying a time point of closure of said aortic valve to coincide with a time point of a maximum in said derivative LAP signal following said minimum in said derivative LAP signal for said cardiac cycle; and
calculating an ejection time parameter value for said subject based on a difference between said time point of opening of said aortic valve and said time point of closure of said aortic valve.

20. The method according to claim 19, further comprising calculating a systolic time ratio parameter value for said subject based on a quotient between said pre-ejection period parameter value and said ejection time parameter value.

21. The method according to claim 20, further comprising calculating an ejection fraction parameter value for said subject as 0.84−0.64×STR, wherein STR denotes said systolic time ratio parameter value.

22. The method according to claim 14, further comprising:
identifying a time point of closure of said aortic valve to coincide with a time point of a maximum in said derivative LAP signal following said minimum in said derivative LAP signal for said cardiac cycle;
identifying a time point of opening of a mitral valve of said heart in said subject to coincide with a time point of a maximum of a V-wave in said LAP signal for said cardiac cycle; and
calculating an isovolumetric relaxation time parameter value for said subject based on a difference between said time point of closure of said aortic valve and said time point of opening of said mitral valve.

23. The method according to claim 14, further comprising:
identifying a time point of closure of a mitral valve of said heart in said subject to coincide with a time point of a maximum of an A-wave in said LAP signal for said cardiac cycle; and
calculating an isovolumetric contraction time parameter value for said subject based on a difference between said time point of closure of said mitral valve and said time point of opening of said aortic valve.

24. The method according to claim 19, further comprising calculating a myocardial performance index parameter value (MPI) for said subject based on a quotient between a sum of said isovolumetric relaxation time parameter value (IRT) and said isovolumetric contraction time parameter value (ICT) and said ejection time parameter value (ET),
$$MPI = \frac{IRT + ICT}{ET} .$$
